(19) United States
(12) Patent Application Publication
(10) Pub. No.: US 2026/0258497 A1
(43) Pub. Date: Sep. 3, 2026

(54) Non-Invasive Successfulness Test of In Vitro Fertilization Process

(71) Applicant: FETUS, IVF INC., Wilmington, DE (US)

(72) Inventors: Miroslava Rabajdová, Košice 1 (SK); Ondrej Slabý, Brno (CZ); Katarína Šoltys, Bratislava (SK); Mária Mareková, Košice 13 (SK); Silvia Toporcerová, Košice (SK)

(21) Appl. No.: 19/365,761

(22) Filed: Oct. 22, 2025

Related U.S. Application Data
(62) Division of application No. 17/905,624, filed on Mar. 1, 2021.

(30) Foreign Application Priority Data
Mar. 2, 2020 (SK) PP 50010-2020

(51) Int. Cl. C12Q 1/6876

(52) U.S. Cl. CPC C12Q 1/6876; C12Q 2600/158; C12Q 2600/178

(57) ABSTRACT

A non-invasive successfulness test of in vitro fertilization process includes two independent steps: the step of identification and analysis of the prognostic canonical and iso-miRNA biomarkers from women's peripheral blood plasma on the day of the IVF process—the Success Test; and the step of prognostic canonical miRNA and iso-miRNA biomarkers from a spent blastocyst medium collected once on day 4 to 6 of the embryo's cultivation—the CultMed Test; wherein the successful Success Test shows the cut off values of the selected miRNA biomarkers at reads number of 1 being ≥1, and the cut off value of miRNA iso-hsa-let-7b-5p_TGAGGTAGTATGTTGTGTGG at reads number of 5 being ≥5, and the successful CultMed Test shows the cut off value of the selected miRNA biomarkers at reads number of 1 being ≥1; and wherein the values in both tests predict the successful in vitro fertilization process.

### CROSS REFERENCES

The present application is a divisional application from co-pending application Ser. No. 17/905,624, which is a National Stage Application of International Application No. PCT/SK2021/050004 filed Mar. 2, 2021, which claims priority to Slovakia Application No. PP 50010-2020 filed Mar. 2, 2020.

### TECHNICAL FIELD

The present teaching relates to the identification of the set of particular miRNA molecules for the determination of IVF (in vitro fertilization) process successfulness/failure, their usage for the creation of a diagnostic test for prediction of in vitro fertilization process successfulness in women during artificial fertilization.

### INCORPORATION-BY-REFERENCE OF SEQUENCE LISTING

This application contains a sequence listing filed in electronic form as an .xml file entitled “SequenceListing.xml” created on Oct. 16, 2025, and has a size of 147,420 bytes. The content of the sequence listing is incorporated herein in its entirety.

### BACKGROUND

Human population health is one of the main attributes of functioning of the human society. High social demands result in many negative effects to overall human health. Presently, 48.5 million couples suffer from infertility worldwide. Considering infertility as a problem of the general population, the number of realized assisted reproduction cycles was significantly increased in the last decades in clinical conditions. In spite of a number of technical improvements in in vitro embryo cultivation, and in spite of all cultivation media improvements, most of the transferred embryos are not able to nestle in the uterus (de Mouzon et al., 2012). Therefore, the relatively low successfulness of the assisted reproduction methods is improved by the transfer of a higher number of embryos within one IVF cycle. Optimal selection of an embryo in the course of IVF is a critical step of the whole treatment. Further to other factors, particularly a high-quality embryo suitable for IVF process contributes to successful implantation. The embryo quality, except for its basic morphological features (monitored by an embryoscope), is not monitored and diagnosed for the determination of IVF process successfulness. Various selection and assessment systems presently used for assessment of human embryos quality have probably achieved their maximum, and they are not able to improve assisted reproduction results. The percentage of IVF process successfulness is about 29%. Therefore, it is necessary to search for another marker. An oocyte itself can predict development of an early embryo in a significant way, because it provides RNA and cellular mechanisms. Many scientific groups study follicular fluid and its proteomics in order to predict the embryos' quality (Kosteria et al., 2017). Collection of the follicular fluid from the individual follicles is, however, technically demanding and a complex procedure in the clinical praxis. Furthermore, the probability of fertilization of the collected oocytes does not reach the required percentage, and therefore more analysis and embryonic methods multiply the economic costs at this level.

Further to the analysis of angiogenesis and vascularization markers, the scientific community focuses on the study of non-coding RNA molecules, i.e. miRNA, which regulate post-transcription gene expression in eukaryotic cells. It was shown that more than 130 miRNA are expressed in the human blastocyst and their expression varies depending on ploidy and gender of the embryos. During embryogenesis the specific miRNA molecules are remarkably expressed. Their levels are detectable in the embryo's cultivation material and also during its in vitro cultivation. The cultivation media become a “waste material” after the completion of the embryo transfer or after freezing of the embryo. Until now several studies dealing with the relationship between non-perceptive and also receptive endometrium in correlation with embryo development competency in the cultivation medium and miRNA (e. g. miR142-5p, miR146a-5p, miR21, miRl9b, miR302c) were published. Nevertheless, no statistically significant and relevant results have been published until now. Discrepancies can be based also on usage of different miRNA analytical methods. Presence/absence of miRNA molecules and their mutual correlation relationship in biologic materials from women in IVF process, and women experiencing repeated implantation failures were not studied until now. It is therefore very needed to create an algorithm for the combination of more miRNA molecules biomarkers, or other non-coding ncRNA, which could eventually determine IVF process successfulness/failure in women. Usage of this information will enable to specify endometrial perceptiveness status, emerging implantation window period, as well as the most suitable time for the embryo transfer in IVF process. Human embryo implementation is a complex and multi-factorial process. miRNAs are endogenous, evolutionary conservative, single stranded non-coding RNA molecules comprising 20-24 nucleotides, which regulate post-transcription gene expression in eukaryotic cells, including mammal cells (Asirvatham et al., 2009; McCallie et al., 2010; Mouillet et al., 2015; Catalanotto et al.,2016). Presently more than 250 human miRNAs are recorded in the miRBase biologic database. The human blastocyst expresses more than 130 miRNAs (McCallie et al., 2010; Rosenbluth et al., 2013, Chan et al., 2018). It was also confirmed that miRNA expression in the human blastocyst varies depending on embryo poidy, as well as on embryo gender (Rosenbluth et al., 2013). miRNA bioinformatics analysis suggests their potential role in the regulation of some biological paths and cell functions, including cell cycle inhibition and tumor suppressing activities (Lee et al., 2014). Biological functions of most miRNAs, however, are not exactly known until now. Several studies investigating the relationship between embryo development competency and miRNAs have been published until now. However, no consensus was established to this day. This discrepancy can be caused by various miRNA analysis methods, times of cultivation medium collection depending on the embryo development day, and a number of other factors. Furthermore, continual synthesis and degradation of miRNA are performed during the division of the early embryo. It was shown that miRNAs are inherited from a mother and up to 60% of miRNAs are lost during the first zygote division (Tang et al., 2007). mRNA expression increases again at blastocyst stage (Zhang et al., 2016). Due to the complexity and the high number of independent processes running during division of the early embryo, it is not probable that one biomarker could predict the embryo development competency in the course of the in vitro fertilization program. It is therefore important to perform studies focusing on the determination of the basic biomarkers combination for the developing embryo before its implantation to uterus, whose combination would correlate with the pregnancy result.

Perceptive endometrium capable of the interaction with the embryo, as well as the determination of the implantation window represent other factors of the multi-factorial IVF process. Presently the non-invasive determination of the endometrium perceptiveness uses particularly ultrasound markers—thickness, volume, and blood flow in the endometrium (Bonilla-Musoles et al., 2013). The implementation window is a time-limited phenomenon in the time-limited secretion phase of menstrual cycle, which is also known as “window of implantation” (IW). Most frequently the implementation window occurs in a short period—between day 20 and day 24 of the ovarian cycle, and it usually lasts only two days. Specific regulation of the expression of selected genes participating in angiogenesis activation, vascularization, and immunity reaction occurs within said period. Present studies directed to the incorrect perceptiveness of the endometrium concluded that different biomarkers' expression, whether increased, or decreased, often results in the perceptiveness disorders. The implantation window is shifted, or, in the worst case, it is eliminated. Due to problematic interpretation and predictive value of the presently available tests, currently the endometrium perceptiveness (ER) tests are used only rarely in infertile women or in women before the planed artificial fertilization (IVF). Implementation failure due to the non-perceptive endometrium remains an unsolved issue of reproduction medicine and a very common reason of infertility in otherwise healthy women. During the last 5 years the percentage rate of the successfully implanted embryos is 29%, in spite of continually increasing efforts to improve the implantation process under in vitro conditions. The selection of the proper embryo, determination of the endometrium perceptiveness, and suitable implantation time in the female cycle are the key variables in the in vitro fertilization process. There are patented tests for endometrium perceptiveness, as well as for the implantation window. Until now, however, no distribution of the particular canonical miRNA and iso-miRNA molecules analyzed by NGS (Next Generation Sequencing) in the determination of the mother's biological competence at multi-factorial consideration of its variation, or IVF process implantation successfulness prediction in the combination with the selection of the high-quality embryo in the specific day of IVF process was patented.

There is no diagnostic test on the world market that enables the prediction of the successfulness of the subsequent IVF process and prognostic selection of the high-quality embryo suitable for the IVF transfer, within 24 hours on the basis of the assessment of the presence of the canonical and iso-miRNA molecules combination in the woman's plasma and in the spent blastocyst medium. The aim of the present teaching is to increase the successfulness of the IVF process in artificial fertilization, and to provide a diagnostic test for the prediction of IVF process successfulness.

### SUMMARY

The present description relates to the identification of the set of the particular miRNA molecules, i.e. the creation of an miRNA profile for the determination of successfulness/failure of the IVF process. Furthermore, the present disclosure relates to the possible of using the miRNA profile for the production of an innovative diagnostic non-invasive test as a personalized medicine product for in vitro fertilization process successfulness prediction in women undergoing artificial fertilization. The diagnostic test includes two independent tests for the identification of prognostic canonic miRNA and iso-miRNA biomarkers. The first one is a Success Test establishing a miRNA profile from peripheral blood plasma of a woman. The second one is a CultMed Test establishing miRNA profile from an embryo's (day 4 to day 6 of its development) cultivation medium. In a standard way the cultivation medium miRNA profile is determined on day 5 of the embryo's development. The spent blastocyst medium can be called as “cultivation waste” or “waste material”.

The present teaching relates to the particular miRNA molecules identified in a woman's plasma, and in a spent blastocyst medium. The specific canonical miRNA and iso-miRNA molecules exhibited significantly different representation and distribution in women's plasma in the group of a successful IVF process in comparison to the women in the group of an unsuccessful IVF process on the particular day. The distribution of the particular canonical miRNA and iso-miRNA molecules (miRNA profile) in plasma can be used for diagnosis, for the assessment of biological competence—readiness or non-readiness of a mother for the IVF process, on the day of the IVF process. Secretion of the particular canonical and iso-miRNA molecules (miRNA profile) to the spent blastocyst medium can be used for diagnosis on the day of IVF process, and for assessment of the embryo's competence, quality.

Accuracy of IVF process prediction was verified by four models as follows:
Logistic regression—a model using a logistic function (Sigmoid) and L2 regularization for binary classification on the basis of the predictive miRNA molecules;
Random Forest—a model using the properties of multiplicatively applied decision trees for binary classification on the basis of the predictive miRNA molecules;
Support Vector Machine (SVM)—a complex model using non-linear method rbftogether with other parameters for binary classification on the basis of the predictive miRNA molecules;
Stochastic gradient descent (SGD)—a model using an iterative randomly initialized logistic regression method together with SGD learning for binary classification on the basis of the predictive miRNA molecules.

The subject matter of the present teaching resides in the non-invasive successfulness test of the IVF (in vitro fertilization) process consisting of two independent steps:
step for identification and analysis of the prognostic canonical and iso-miRNA biomarkers from the peripheral blood plasma of women on the day of the IVF process (Success Test), and
step for prognostic canonical miRNA and iso-miRNA biomarkers from the spent blastocyst medium collected once on day 4 to 6 of the embryo cultivation (CultMed Test),
wherein the successful Success Test shows the cut off values, for the selected miRNA biomarkers at reads number of 1, that are ≥1, and the cut off value, for miRNA iso-hsa-let-7b-5p_TGAGGTAGTATGTTGTGTGG at reads number of 5, that is ≥5, and
the successful CultMed Test shows the cut off value of the selected miRNA biomarkers at reads number of 1, being ≥1, and wherein these values of both tests predicate the successful IVF process.

One analysis of the non-invasive IVF successfulness test includes the identification of the prognostic canonical miRNA and iso-miRNA biomarkers—Success Test with 100% sensitivity and 100% specificity having average accuracy of 4 different models of 88%, performed in the following steps:
Taking a woman's peripheral blood plasma sample.
Isolating all miRNA followed by performing sequence analysis, and data analysis for selected 19 miRNA: iso-hsa-miR-126-3p with SEQ ID NO: 1, iso-hsa-let-7i-5p with SEQ ID NO: 2, iso-hsa-miR-342-3p with SEQ ID NO: 3, iso-hsa-let-7b-5p with SEQ ID NO: 4, iso-hsa-miR-132-3p with SEQ ID NO: 5, iso-hsa-miR-23b-3p with SEQ ID NO: 6, iso-hsa-miR-125a-5p with SEQ ID NO: 7, iso-hsa-miR-584-5p with SEQ ID NO: 8, iso-hsa-miR-127-3p with SEQ ID NO: 9, iso-hsa-miR-101-3p with SEQ ID NO: 10, iso-hsa-miR-151a-3p with SEQ ID NO: 11, iso-hsa-miR-92a-3p with SEQ ID NO: 12, iso-hsa-let-7b-5p with SEQ ID NO: 13, iso-hsa-miR-589-5p with SEQ ID NO: 14, iso-hsa-miR-652-3p with SEQ ID NO: 15, iso-hsa-miR-1307-3p with SEQ ID NO: 16, iso-hsa-miR-382-5p with SEQ ID NO: 17, iso-hsa-let-7b-5p with SEQ ID NO: 18, iso-hsa-let-7i-5p with SEQ ID NO: 19.
Determining the reads number for 19 miRNA biomarkers from the previous step.

The second part of non-invasive IVF successfulness test analysis includes the identification of the prognostic canonical and iso-miRNA biomarkers—CultMed Test with 100% sensitivity and 80% specificity having average accuracy of 4 different models of 85%, performed in the following steps:
Taking a spent blastocyst medium sample.
Isolating all miRNA followed by performing sequence analysis, and data analysis for selected 17 miRNA: iso-hsa-let-7b-5p with SEQ ID NO: 20, iso-hsa-miR-423-5p with SEQ ID NO: 21, iso-hsa-miR-21-5p with SEQ ID NO: 22, iso-hsa-miR-486-5p with SEQ ID NO: 23, iso-hsa-miR-486-5p with SEQ ID NO: 24, iso-hsa-miR-25-3p with SEQ ID NO: 25, iso-hsa-miR-3613-5p with SEQ ID NO: 26, iso-hsa-miR-142-3p with SEQ ID NO: 27, iso-hsa-miR-664a-5p with SEQ ID NO: 28, iso-hsa-miR-151a-3p with SEQ ID NO: 29, iso-hsa-miR-92a-3p with SEQ ID NO: 30, iso-hsa-let-7a-5p with SEQ ID NO: 31, iso-hsa-let-7b-5p with SEQ ID NO: 32, iso-hsa-miR-100-5p with SEQ ID NO: 33, iso-hsa-miR-191-5p with SEQ ID NO: 34, iso-hsa-let-7a-5p with SEQ ID NO: 35, iso-hsa-let-7d-5p with SEQ ID NO: 36.
Determining the reads number for 17 miRNA biomarkers from the previous step.

Test for the analysis of the identification of the prognostic canonical miRNA and iso-miRNA biomarkers, the Success Test having 99% accuracy of AUC ROC prediction curve model includes:
Taking the woman's plasma sample.
Isolating all miRNA followed by performing sequence analysis, and data analysis for selected 10 miRNA: iso-hsa-miR-126-3p, iso-hsa-let-7i-5p, iso-hsa-miR-342-3p, iso-hsa-let-7b-5p, iso-hsa-miR-132-3p, iso-hsa-miR-23b-3p, iso-hsa-miR-125a-5p, hsa-miR-584-5p, iso-hsa-miR-127-3p, iso-hsa-miR-151a-3p.
Determining the reads number for 10 miRNA biomarkers from the previous step.

Test for the analysis of the identification of the prognostic canonical and iso-miRNA biomarkers, the CultMed Test having 95% accuracy of AUC ROC prediction curve model includes:
Taking the spent blastocyst medium sample.
Isolating all miRNA followed by performing sequence analysis, and data analysis for the first selected 14 miRNA: iso-hsa-let-7b-5p, iso-hsa-miR-423-5p, iso-hsa-miR-21-5p, iso-hsa-miR-486-5p, iso-hsa-miR-486-5p, iso-hsa-miR-25-3p, iso-hsa-miR-3⁶¹³-5p, iso-hsa-miR-142-3p, iso-hsa-miR-664a-5p, iso-hsa-miR-151a-3p, iso-hsa-miR-92a-3p, iso-hsa-let-7a-5p, iso-hsa-let-7b-5p, iso-hsa-miR-100-5p.
Determining the reads number for 14 miRNA biomarkers from the previous step.

The non-invasive test by the Success Test predicates the IVF process successfulness at cut off value of 1, while the selected miRNA molecules having value of ≥1 predict successful IVF process, and ones having value of <1 predict unsuccessful IVF process. The cut off value of 5 classifies the selected miRNA molecules having the value of ≥5 as the molecules predicting successful IVF process, and ones having the values of <5 as predicting IVF process failure. Non-invasive test method via CultMed Test predicts the IVF process successfulness at the cut off value of 1, while miRNA molecules having the value of ≥1 predict a high competent (high-quality) embryo, and ones having the value of <1 predict a low competent (low-quality) embryo.

In the next step of the non-invasive test the identified miRNA molecules form the woman's plasma are arranged according to their significance for the test result: iso-hsa-miR-126-3p, hsa-let-7i-5p, iso-hsa-miR-342-3p, iso-hsa-let-7b-5p, iso-hsa-miR-132-3p, iso-hsa-miR-23b-3p, iso-hsa-miR-125a-5p, iso-hsa-miR-584-5p, iso-hsa-miR-127-3p, iso-hsa-miR-101-3p, iso-hsa-miR-151a-3p, iso-hsa-miR-92a-3p, iso-hsa-let-7b-5p, iso-hsa-miR-589-5p, iso-hsa-miR-652-3p, iso-hsa-miR-1307-3p, iso-hsa-miR-382-5p, iso-hsa-let-7b-5p, iso-hsa-let-7i-5p.

In the next step of the non-invasive test the identified miRNA molecules from the spent blastocyst medium are arranged according to their significance for the test result: iso-hsa-let-7b-5p, iso-hsa-miR-423-5p, iso-hsa-miR-21-5p, iso-hsa-miR-486-5p, iso-hsa-miR-486-5p, iso-hsa-miR-25-3p, iso-hsa-miR-3⁶¹³-5p, iso-hsa-miR-142-3p, iso-hsa-miR-664a-5p, iso-hsa-miR-151a-3p, iso-hsa-miR-92a-3p, iso-hsa-let-7a-5p, iso-hsa-let-7b-5p, iso-hsa-miR-100-5p, iso-hsa-miR-191-5p, iso-hsa-let-7a-5p, iso-hsa-let-7d-5p.

The advantage of the diagnostic test is the availability of the result for IVF process successfulness/failure within 24 hours from the sampling of the biological material. The main subjects of the present teaching were performed using the method of massive-parallel sequencing of non-coding small RNA molecules (miRNA) at Illumina sequencing platform. On the basis of the specific data analysis (Explanatory Data Analysis) and Machine Learning methods with several models, a number of predictive biomarkers for the individual biological material types were selected.

A list of the particular canonical miRNA and iso-miRNA molecules identified from women plasmas and canonical and iso-miRNA molecules identified from the spent blastocyst media represent another fundamental part of the present teaching. miRNA molecules detected by the Success Test are shown in Table 1.

TABLE 1
Biomarkers for miRNA prediction of the IVF process successfulness/failure

| | | p |
|---|---|---|
| miRNA biomarkers for the | Coef | (p-value, H0: data |
| prediction of IVF process | (Coefficient of | distribution in relation to |
| successfulness/failure | significance) | IVF result is identical) |
| iso-hsa-miR-126- | 11.520844189682128 | 0.0016884300209033492 |
| 3p_CGTACCGTGAGAAATAATGCGT | | |
| iso-hsa-let-7i- | 10.851674641148328 | 0.002221447605170916 |
| 5p_TGTGGTAGTAGTTTGTGCTGTA | | |
| iso-hsa-miR-342- | 10.765113798008537 | 0.0023025382184651104 |
| 3p_TCTCACACAGAAATCGGACCCGTCT | | |
| iso-hsa-let-7b- | 10.547046485537292 | 0.002521143336464067 |
| 5p_TGAGGTAGTAGGTTGTGTGTTA | | |
| iso-hsa-miR-132- | 9.910931174089068 | 0.0032951920974540376 |
| 3p_TAACAGCCTACAGCCATGGTCGT | | |
| iso-hsa-miR-23b- | 9.373481781376519 | 0.0041475542794915604 |
| 3p_GCACATTGCCAGGGATTACCACT | | |
| iso-hsa-miR-125a- | 9.18292128014561 | 0.004503999211390216 |
| 5p_TCCCTGAGACCTITTAACCTGT | | |
| iso-hsa-miR-584- | 9.094736842105265 | 0.004679908836274879 |
| 5p_TATGGTTTGCCTGGGACTG | | |
| iso-hsa-miR-127- | 9.094736842105265 | 0.004679908836274879 |
| 3p_TCGGATCAGTCTGAGCTTGGCTTTT | | |
| iso-hsa-miR-101- | 8.845594649607445 | 0.005217803371020453 |
| 3p_TACACTACTGTGATAACTGA | | |
| iso-hsa-miR-151a- | 8.845594649607445 | 0.005217803371020453 |
| 3p_TACTAGACTAAAGCTCCTTGAGGT | | |
| iso-hsa-miR-92a- | 8.845594649607445 | 0.005217803371020453 |
| 3p_GATTGCACTTGTCCCGGCCTGAA | | |
| iso-hsa-let-7b- | 8.816195372750641 | 0.005285512415951764 |
| 5p_TGAGGTAGTATGTTGTGTGG | | |
| iso-hsa-miR-589- | 8.696842105263158 | 0.0055702068262932485 |
| 5p_TGAGAACCACGTCCGCTCTGAGC | | |
| iso-hsa-miR-652- | 8.418140050047175 | 0.0063009456943287695 |
| 3p_GATGGCGCCACTAGGGTTGTGA | | |
| iso-hsa-miR-1307- | 8.418140050047175 | 0.0063009456943287695 |
| 3p_ACTCGGCGTAGCGTCGGTCGTGT | | |
| iso-hsa-miR-382- | 8.289473684210527 | 0.006672365487531239 |
| 5p_GAAGTTGTTCGCGGTGGATTC | | |
| iso-hsa-let-7b- | 8.289473684210527 | 0.006672365487531239 |
| 5p_TGAGGTAGTAAGTTGTGTGGTAT | | |
| iso-hsa-let-7i- | 8.289473684210527 | 0.006672365487531239 |
| 5p_TGAGTTAGTAGTTTGTGCTGTTTT | | |

miRNAs in Table 1 are ordered according to their significance coefficients, which coefficients determine how important is it to deal with a specific molecule in the analysis (what is its role in the result prediction).

TABLE 2
Threshold AUC ROC (Receiver operating characteristic of Logistic
Regression), miRNA biomarkers sensitivity and specificity predicting IVF process
successfulness/failure in women plasmas

| Threshold, Cut off values for | | | | | |
|---|---|---|---|---|---|
| miRNA biomarkers for the IVF | | | | | |
| process successfulness/failure | | Sensitivity | Specificity | | |
| predictions | Threshold | % | % | AUC | CoffNC |
| iso-hsa-miR-126- | 0.7 | 81 | 35 | 0.75 | 1 |
| 3p_CGTACCGTGAGAAATAATGCGT | | | | | |
| iso-hsa-let-7i- | 0.608 | 1 | 64 | 0.68 | 1 |
| 5p_TGTGGTAGTAGTTTGTGCTGTA | | | | | |
| iso-hsa-miR-342- | 0.768 | 52 | 58 | 0.74 | 1 |
| 3p_TCTCACACAGAAATCGGACCCGTCT | | | | | |
| iso-hsa-let-7b- | 0.651 | 91 | 47 | 0.65 | 1 |
| 5p_TGAGGTAGTAGGTTGTGTGTTA | | | | | |
| iso-hsa-miR-132- | 0.81 | 38 | 0 | 0.69 | 1 |
| 3p_TAACAGCCTACAGCCATGGTCGT | | | | | |
| iso-hsa-miR-23b- | 0.61 | 100 | 65 | 0.68 | 1 |
| 3p_GCACATTGCCAGGGATTACCACT | | | | | |
| iso-hsa-miR-125a- | 0.75 | 48 | 58 | 0.71 | 1 |
| 5p_TCCCTGAGACCTITTAACCTGT | | | | | |
| iso-hsa-miR-584- | 0,62 | 100 | 65 | 0.68 | 1 |
| 5p_TATGGTTTGCCTGGGACTG | | | | | |
| iso-hsa-miR-127- | 0.61 | 100 | 65 | 0.68 | 1 |
| 3p_TCGGATCAGTCTGAGCTTGGCTTTT | | | | | |
| iso-hsa-miR-101- | 0.621 | 95 | 59 | 0.68 | 1 |
| 3p_TACACTACTGTGATAACTGA | | | | | |
| iso-hsa-miR-151a- | 0.62 | 95 | 59 | 0.68 | 1 |
| 3p_TACTAGACTAAAGCTCCTTGAGGT | | | | | |
| iso-hsa-miR-92a- | 0.62 | 95 | 59 | 0.69 | 1 |
| 3p_GATTGCACTTGTCCCGGCCTGAA | | | | | |
| iso-hsa-let-7b- | 0.67 | 67 | 12 | 0.78 | 5 |
| 5p_TGAGGTAGTATGTTGTGTGG | | | | | |
| iso-hsa-miR-589- | 0.80 | 38 | 0 | 0.69 | 1 |
| 5p_TGAGAACCACGTCCGCTCTGAGC | | | | | |
| iso-hsa-miR-652- | 0,62 | 95 | 59 | 0.68 | 1 |
| 3p_GATGGCGCCACTAGGGTTGTGA | | | | | |
| iso-hsa-miR-1307- | 0.63 | 95 | 58 | 0.68 | 1 |
| 3p_ACTCGGCGTAGCGTCGGTCGTGT | | | | | |
| iso-hsa-miR-382- | 0.59 | 100 | 71 | 0.65 | 1 |
| 5p_GAAGTTGTTCGCGGTGGATTC | | | | | |
| iso-hsa-let-7b- | 0.60 | 100 | 71 | 0.72 | 1 |
| 5p_TGAGGTAGTAAGTIGTGTGGTAT | | | | | |
| iso-hsa-let-7i- | 0.60 | 100 | 70 | 0.65 | 1 |
| 5p_TGAGTTAGTAGTTTGTGCTGTTTT | | | | | |

Cut off value of the normalized reads (CoffNC), CoffNC ABS (TPR-(1-FPR), value ≥ CoffNC = successful IVF; value < CoffNC = IVF failure

For each of the selected miRNA molecules the minimal number of sequencing reads stated in the last column in Table 2 is needed (cut off value of the normalized reads=1 or 5). If a miRNA molecule has its cut off value determined as 1, then all values ≥1 predict successful IVF process, and miRNA molecules cut off values <1 predict IVF process failure. The cut off value for the normalized reads determined for iso-hsa-let-7b-5p molecule is 5. It means that any value ≥5 predicts a successful IVF process, and any value <5 predicts IVF process failure.

For 100% prediction of IVF process successfulness/failure it is necessary to analyze each of 19 selected biomarker miRNA molecules (Table 2). Also, lower number of miRNA molecules can be used for the analysis of IVF process successfulness/failure, however, the result prediction accuracy will be lower.

The Success Test can be used as safe, non-invasive diagnostic test of IVF process successfulness. It is possible to make a commercial product (service) intended for the public on the basis of this test.

Said canonical and iso-miRNA molecules having the above sequences can be used also in other forms of diagnostic tests as specific probes for the determination of IVF process successfulness.

Table 3 shows the list of the particular canonical and iso-miRNA molecules detected from the spent blastocyst media by the CultMed Test. The molecules identified in the spent blastocyst medium are ordered according to their importance, i.e. significance on the basis of the significance coefficient determining how important it is to deal with a given molecule in the analysis (how significant is its role in result prediction).

TABLE 3
Prediction biomarkers for the selection of competent embryo/incompetent embryo in
the IVF process

| | | p |
|---|---|---|
| Prediction biomarkers for the | | (p-value, H0: data |
| selection of competent | coef | distribution in relation |
| embryo/incompetent embryo in | (Coefficient of | to IVF result is |
| IVF process | significance) | identical) |
| iso-hsa-let-7b- | 9.621526827042178 | 0.003391088823611076 |
| 5p_TGAGGTGGTAGGTTGTGTGGT | | |
| iso-hsa-miR-423- | 9.529546351084809 | 0.003532537765767483 |
| 5p_TGAGGGGCAGAGAGCGAGACTTA | | |
| iso-hsa-miR-21- | 9.37945619335347 | 0.003776937060541304 |
| 5p_TAGCTTACCAGACTGATGTTGAC | | |
| iso-hsa-miR-486- | 8.899093803575798 | 0.004687231604519787 |
| 5p_TCCTGTACTGAGCTGCCCAGAGA | | |
| iso-hsa-miR-486- | 7.886037735849056 | 0.007462265245605795 |
| 5p_TCCTGTACTGAGCTGCCTCGAG | | |
| iso-hsa-miR-25- | 7.166666666666666 | 0.010471649328310581 |
| 3p_CATTGCAATTGTCTCGGTCTGA | | |
| iso-hsa-miR-3613- | 6.723975915506861 | 0.01294940775142319 |
| 5p_TGTTGTACTTTTTTTTTTGTT | | |
| iso-hsa-miR-142- | 6.254545454545453 | 0.0162767044425456 |
| 3p_TGTAGTGTTTCCTACCTTATGGA | | |
| iso-hsa-miR-664a- | 6.254545454545453 | 0.0162767044425456 |
| 5p_ACTGGCTAGGGAAAATGATTGGA | | |
| iso-hsa-miR-151a- | 5.757024793388432 | 0.020828127625493652 |
| 3p_CAAGACTGAAGCTCCTTGAGG | | |
| iso-hsa-miR-92a- | 5.752285490865144 | 0.020877563509075946 |
| 3p_TATCGCACTTGTCCCGGCCTGT | | |
| iso-hsa-let-7a- | 5.676567656765675 | 0.021684740586716857 |
| 5p_TGATGTAGTAGGTTGTATAG | | |
| iso-hsa-let-7b- | 5.618093452752898 | 0.022331072190788642 |
| 5p_TGAGGTAGTAGGTTGTGTGGTTTA | | |
| iso-hsa-miR-100- | 5.448383233532933 | 0.024327313909886367 |
| 5p_AACCCGTAGATCCGAACTTGT | | |
| iso-hsa-miR-191- | 5.3960784313725485 | 0.024980707010107902 |
| 5p_CAATGGAATCCCAAAAGCAGCTG | | |
| iso-hsa-let-7a- | 5.3960784313725485 | 0.024980707010107902 |
| 5p_TGAGTTAGTAGGTTGTATAG | | |
| iso-hsa-let-7d | 5.3960784313725485 | 0.024980707010107902 |
| 5p_AGAGGTAGTAGGTTGCGTAGTT | | |

17 prediction biomarkers classified as significant prediction biomarkers for the determination of an embryo competence in the IVF process were selected on the basis of the machine learning methods.

TABLE 4
Threshold AUC ROC, sensitivity and specificity of miRNA biomarkers predicting
a high-quality embryo and a low quality embryo

| Threshold, Cut off values for | | | | | |
|---|---|---|---|---|---|
| miRNA biomarkers of IVF | | | | | |
| process successfulness/ | | Sensitivity | Specificity | | |
| failure predictions | Threshold | % | % | AUC | CoffNC |
| iso-hsa-let-7b- | 0.5 | 92 | 47 | 70 | 1 |
| 5p_TGAGGTGGTAGGTTGTGTGGT | | | | | |
| iso-hsa-miR-423- | 0.5 | 92 | 43 | 68 | 1 |
| 5p_TGAGGGGCAGAGAGCGAGACTTA | | | | | |
| iso-hsa-miR-21- | 0.4 | 96 | 38 | 67 | 1 |
| 5p_TAGCTTACCAGACTGATGTTGAC | | | | | |
| iso-hsa-miR-486- | 0.4 | 96 | 38 | 67 | 1 |
| 5p_TCCTGTACTGAGCTGCCCAGAGA | | | | | |
| iso-hsa-miR-486- | 0.4 | 96 | 38 | 67 | 1 |
| 5p_TCCTGTACTGAGCTGCCTCGAG | | | | | |
| iso-hsa-miR-25- | 0.3 | 100 | 24 | 62 | 1 |
| 3p_CATTGCAATTGTCTCGGTCTGA | | | | | |
| iso-hsa-miR-3613- | 0.6 | 96 | 29 | 63 | 1 |
| 5p_TGTTGTACTTTTTTTTTTGTT | | | | | |
| iso-hsa-miR-142- | 0.3 | 100 | 24 | 62 | 1 |
| 3p_TGTAGTGTTTCCTACCTTATGGA | | | | | |
| iso-hsa-miR-664a- | 0.3 | 100 | 24 | 62 | 1 |
| 5p_ACTGGCTAGGGAAAATGATTGGA | | | | | |
| iso-hsa-miR-151a- | 0.5 | 50 | 81 | 68 | 1 |
| 3p_CAAGACTGAAGCTCCTTGAGG | | | | | |
| iso-hsa-miR-92a- | 0.5 | 96 | 29 | 63 | 1 |
| 3p_TATCGCACTTGTCCCGGCCTGT | | | | | |
| iso-hsa-let-7a- | 0.3 | 100 | 24 | 62 | 1 |
| 5p_TGATGTAGTAGGTTGTATAG | | | | | |
| iso-hsa-let-7b- | 0.6 | 33 | 95 | 65 | 1 |
| 5p_TGAGGTAGTAGGTTGTGTGGTTTA | | | | | |
| iso-hsa-miR-100- | 0.6 | 30 | 95 | 63 | 1 |
| 5p_AACCCGTAGATCCGAACTTGT | | | | | |
| iso-hsa-miR-191- | 0.3 | 100 | 19 | 60 | 1 |
| 5p_CAATGGAATCCCAAAAGCAGCTG | | | | | |
| iso-hsa-let-7a- | 0.3 | 100 | 19 | 60 | 1 |
| 5p_TGAGTTAGTAGGTTGTATAG | | | | | |
| iso-hsa-let-7d | 0.3 | 100 | 19 | 60 | 1 |
| 5p_AGAGGTAGTAGGTTGCGTAGTT | | | | | |

Cut off value of the normalized reads (CoffNC); CoffNC ABS (TPR-(1-FPR), value ≥ CoffNC = a high-quality embryo; value < CoffNC = a low-quality embryo

The last column of Table 4 states the cut off value of normalized reads. If a miRNA molecule has its cut off value determined as 1, then all values ≥1 predict a high-quality/high competent embryo, and miRNA molecules cut off values <1 predict a low-quality/low-competent embryo. On the basis of these values, it is possible to select a high-quality embryo suitable for the transfer in the IVF process.

All 17 selected miRNA biomarkers are needed for the prediction analysis of high-competent/low-competent embryo with 100% sensitivity and 80% specificity. Also, lower number of miRNA molecules can be used for the analysis of a high-competent/low-competent embryo; however, the result prediction accuracy will be lower.

The CultMed Test can be also used as the safe, non-invasive diagnostic test for the selection of a high-competent embryo. This test can be made as a diagnostic product (service); however, it can be also used for the selection of an embryo, which is the most suitable for the IVF transfer (as a complementary test to the Success Test).

Said canonical and iso-miRNA molecules having the above sequences can be used also in other forms of diagnostic tests as specific probes for the selection of an embryo, which is the most suitable for the IVF transfer.

The tests are independent, and it is suitable to combine them, in order to increase IVF process successfulness, due to the optimization of IVF term prediction in a patient, as well as more effective embryo selection. Said diagnostic test can be offered to infertile couples as a service—a mother competence diagnosis (selection of the most suitable IVF transfer day), an embryo selection.

### BRIEF DESCRIPTION OF DRAWINGS

FIG. 1—Resulting ROC curve based on the analysis of 19 miRNA biomarkers from woman's plasma

FIG. 2—Resulting ROC curve based on the analysis of 10 miRNA biomarkers from woman's plasma

FIG. 3—Resulting ROC curve based on the analysis of 17 miRNA biomarkers from a spent blastocyst medium

FIG. 4—Resulting ROC curve based on the analysis of 14 miRNA biomarkers from a spent blastocyst medium

### EXAMPLES OF THE EMBODIMENT

### Example 1

Example 1 describes the detection of the particular canonical miRNA and iso-miRNA molecules isolated from peripheral blood plasma of 37 women in the age under 37 years by NGS sequencing method. At the day of the embryo transfer, peripheral blood was collected from the women, which blood was used for plasma non-coding RNA molecules analysis. 19 selected canonical miRNA and iso-miRNA molecules were analyzed (Table 2).

### Evaluation:

In the tested plasma the cut off values for miRNA biomarker were at the level ≥1. In case of iso-hsa-let-7b-5p biomarker the cut off value was ≥5.

The remaining cut off values were <1.

The successful IVF process was achieved in 20 women and IVF process failure was observed in 17 women.

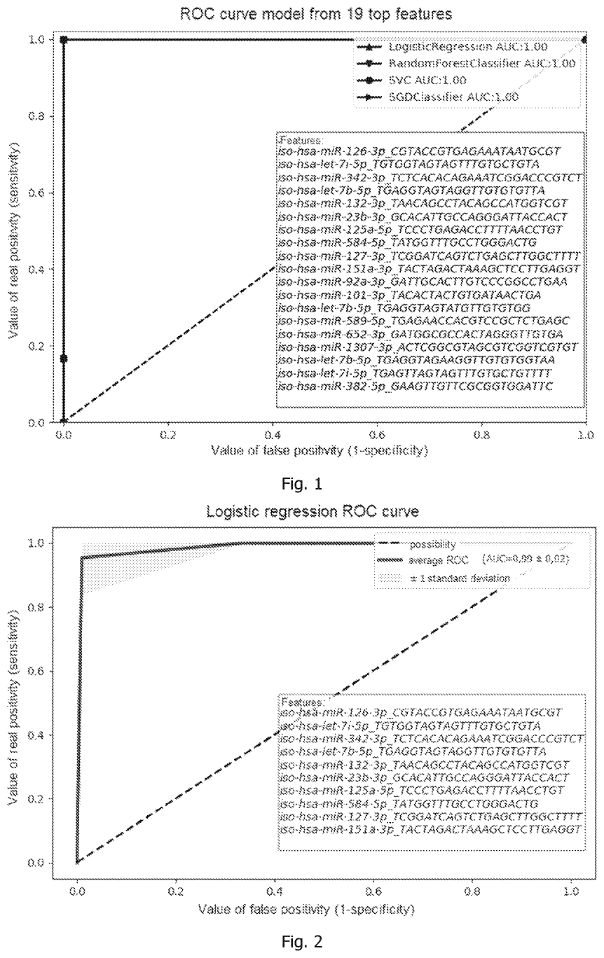

ROC analysis (Receiver Operating Characteristic curve) showed that the combination of 19 canonical miRNA molecules isolated from the women's plasma on the embryo transfer day (see Table 2) predict the IVF process successfulness/failure with 100% sensitivity and 100% specificity (Area Under the ROC curve, AUC=1,0, [1-1]; p=0.001), as illustrated in FIG. 1.

### Example 2

Example 2 describes the detection of the particular canonical miRNA and iso-miRNA molecules detected in plasma of 37 women in the age under 37 years by NGS sequencing method. On the day of the embryo transfer, peripheral blood was collected from the women, whose blood was used for plasma non-coding RNA molecules analysis. Lower number of the canonical miRNA was used for the analysis. 10 canonical miRNA and iso-miRNA as stated in the following table were selected:

miRNA biomarkers for the prediction of
IVF process successfulness/failure

iso-hsa-miR-126-
3p_CGTACCGTGAGAAATAATGCGT
iso hsa-let-7i-
5p_TGTGGTAGTAGTTTGTGCTGTA
iso-hsa-miR-342-
3p_TCTCACACAGAAATCGGACCCGTCT
iso-hsa-let-7b-
5p_TGAGGTAGTAGGTTGTGTGTTA
iso-hsa-miR-132-
3p_TAACAGCCTACAGCCATGGTCGT
iso-hsa-miR-23b-
3p_GCACATTGCCAGGGATTACCACT
iso-hsa-miR-125a-
5p_TCCCTGAGACCTITTAACCTGT
iso-hsa-miR-584-5p_TATGGTTTGCCTGGGACTG
iso-hsa-miR-127-
3p_TCGGATCAGTCTGAGCTTGGCTTTT
iso-hsa-miR-151a-
3p_TACTAGACTAAAGCTCCTTGAGGT

### Evaluation:

The method of the test samples and confirmation samples permutation was used in case of the change of the significant biomarkers number, with 7 repetitions and 20/80 percent ratio. ROC method analysis showed that the combination of 10 canonical miRNA molecules collected from the women's plasma on the embryo transfer day (FIG. 2) predicts the IVF process successfulness/failure. Average value of the prediction accuracy measured by AUC calculation is at the level of 0.97 (resp. 97%), with variation+/−0.06 (FIG. 2). The analysis of 10 canonical miRNAs (from plasma) has the prediction accuracy for the test result at the level of 97%.

### Example 3

Example 3 describes the detection of the particular canonical and iso-hsa-miRNA molecules isolated from the cultivation media of 45 embryos. The cultivation medium was standardly collected on day 5 of the embryo's cultivation. The embryo secreted non-coding RNA molecules to the cultivation fluid, whose molecules are used for the differential selection of a high-quality embryo. 17 iso-hsa-miRNA molecules sequenced by NGS method were selected from a number of the molecules detected in the spent blastocyst medium (Table 4). The number of miRNA biomarkers predicting a high-quality/low-quality embryo was determined by the machine learning methods.

### Evaluation:

The cut off values of the cultivation media of the high-competent embryos were ≥1 for all tested miRNA biomarkers.

The cut off values of the cultivation media of the low-competent embryos were <1 for all tested miRNA biomarkers.

On the basis of the results the spent blastocyst media were divided into two groups: 25 cultivation media showed the high-competent/high-quality embryo, 20 cultivation media showed the low-competent/low-quality embryo.

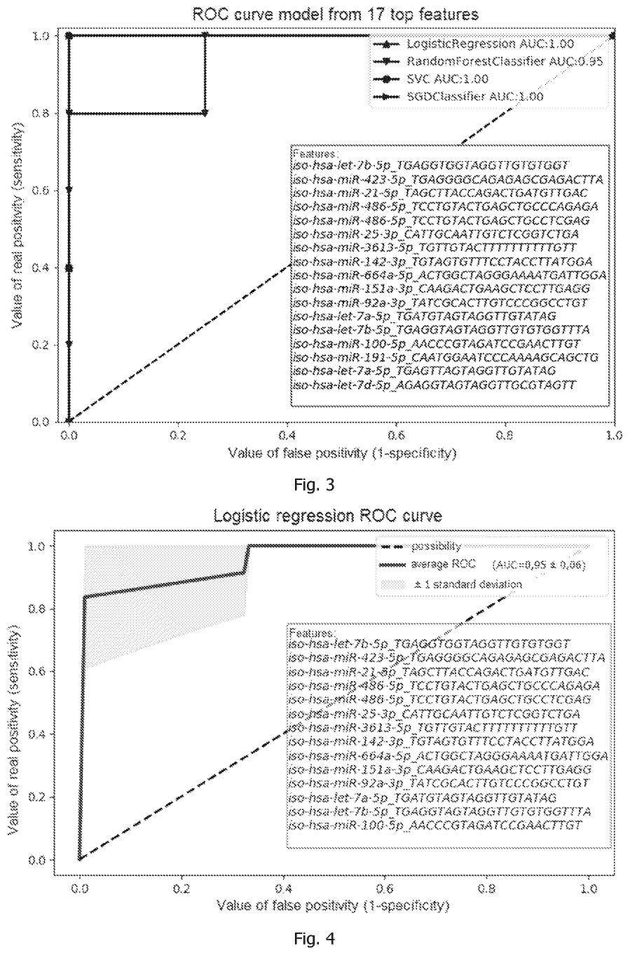

ROC analysis showed that 17 canonical and iso-miRNA molecules (Table 4) distinguish, predict, differentiate a high-competent embryo from a low-competent (non-successful) embryo in the IVF process with 100% sensitivity and 80% specificity (AUC=0.9 [0.8-1.0]; p=0.001). ROC is illustrated in FIG. 3.

### Example 4

The protocol was the same as in Example 3, however 14 canonical and iso-miRNA molecules isolated from the spent blastocyst media were selected for the analysis of a high-competent/low-competent embryo, and these molecules are shown in the following table:

Prediction biomarkers for the selection of
competent/incompetent embryo in IVF
process

iso-hsa-let-7b-5p_TGAGGTGGTAGGTTGTGTGGT
iso-hsa-miR-423-
5p_TGAGGGGCAGAGAGCGAGACTTA
iso-hsa-miR-21-5p_TAGCTTACCAGACTGATGTTGAC
iso-hsa-miR-486-
5p_TCCTGTACTGAGCTGCCCAGAGA
iso-hsa-miR-486-5p_TCCTGTACTGAGCTGCCTCGAG
iso-hsa-miR-25-3p_CATTGCAATTGTCTCGGTCTGA
iso-hsa-miR-3613-5p_TGTTGTACTTTTTTTTTTGTT
iso-hsa-miR-142-3p_TGTAGTGTTTCCTACCTTATGGA
iso-hsa-miR-664a-
5p_ACTGGCTAGGGAAAATGATTGGA
iso-hsa-miR-151a-3p_CAAGACTGAAGCTCCTTGAGG
iso-hsa-miR-92a-3p_TATCGCACTTGTCCCGGCCTGT
iso-hsa-let-7a-5p_TGATGTAGTAGGTTGTATAG
iso-hsa-let-7b-5p_TGAGGTAGTAGGTTGTGTGGTTTA
iso-hsa-miR-100-5p_AACCCGTAGATCCGAACTTGT

### Evaluation:

As in Example 2, the method of the test samples and confirmation samples permutation was used with 7 repetitions and 20/80 percent ratio. Said ROC analysis method showed that the canonical and iso-miRNA molecules illustrated in FIG. 4 distinguish, differentiate a high-competent embryo from a low-competent (non-successful) embryo in the IVF process with lower number of the prediction mRNA biomarkers (14). Average value of the prediction accuracy measured by AUC calculation is at the level of 0.97 (resp. 97%), with variation+/−0.06 (FIG. 4). The analysis of 14 canonical and iso-miRNAs from the spent blastocyst medium provides the prediction accuracy for the test result at the level of 97%.

1. (canceled)

2. (canceled)

3. (canceled)

4. (canceled)

5. (canceled)

6. (canceled)

7. (canceled)

8. A method for the evaluation of in vitro fertilization process successfulness, comprises two independent analyses:
identification and analysis of selected prognostic canonical miRNA and/or iso-miRNA biomarkers from women's peripheral blood plasma on the day of the in vitro fertilization process;
wherein successful evaluation shows cut off values ≥1 at reads number of 1, and cut off values ≥5 at reads number of 5;
identification and analysis of selected prognostic canonical miRNAs and/or iso-miRNAs from a spent blastocyst medium collected once on day 4 to 6 of the embryo's cultivation with 100% sensitivity and 80% specificity comprises the following:
i) taking the spent blastocyst medium sample;
ii) isolating all miRNA and/or iso-miRNA biomarkers, followed by performing sequence analysis, and the data analysis for the selected 17 miRNA and/or iso-miRNA biomarkers ordered according to their significance for the in vitro fertilization process result:
iso-hsa-let-7b-5p in SEQ ID NO: 20,
TGAGGTGGTAGGTTGTGTGGT
iso-hsa-miR-423-5p in SEQ ID NO: 21,
TGAGGGGCAGAGAGCGAGACTTA
iso-hsa-miR-21-5p in SEQ ID NO: 22,
TAGCTTACCAGACTGATGTTGAC
iso-hsa-miR-486-5p in SEQ ID NO: 23,
TCCTGTACTGAGCTGCCCAGAGA
iso-hsa-miR-486-5p in SEQ ID NO: 24,
TCCTGTACTGAGCTGCCTCGAG
iso-hsa-miR-25-3p in SEQ ID NO: 25,
CATTGCAATTGTCTCGGTCTGA
iso-hsa-miR-3613-5p in SEQ ID NO: 26,
TGTTGTACTTTTTTTTTTGTT
iso-hsa-miR-142-3p in SEQ ID NO: 27,
TGTAGTGTTTCCTACCTTATGGA
iso-hsa-miR-664a-5p in SEQ ID NO: 28,
ACTGGCTAGGGAAAATGATTGGA
iso-hsa-miR-151a-3p in SEQ ID NO: 29,
CAAGACTGAAGCTCCTTGAGG
iso-hsa-miR-92a-3p in SEQ ID NO: 30,
TATCGCACTTGTCCCGGCCTGT
iso-hsa-let-7a-5p in SEQ ID NO: 31,
TGATGTAGTAGGTTGTATAG
iso-hsa-let-7b-5p in SEQ ID NO: 32,
TGAGGTAGTAGGTTGTGTGGTTTA
iso-hsa-miR-100-5p in SEQ ID NO: 33,
AACCCGTAGATCCGAACTTGT
iso-hsa-miR-191-5p in SEQ ID NO: 34,
CAATGGAATCCCAAAAGCAGCTG
iso-hsa-let-7a-5p in SEQ ID NO: 35,
TGAGTTAGTAGGTTGTATAG
iso-hsa-let-7d 5p in SEQ ID NO: 36,
AGAGGTAGTAGGTTGCGTAGTT
iii) determining the reads number for all 17 miRNA and/or iso-miRNA biomarkers from ii);
wherein the successful evaluation shows cut off values ≥1 at reads number of 1; and
wherein if the evaluation of both analyses is successful, then also the prediction of in vitro fertilization process is successful.

9. The method according to claim 8, wherein the evaluation of the identification of the selected prognostic canonical miRNA and iso-miRNA biomarkers from the women's peripheral blood plasma on the day of the in vitro fertilization process with 100% sensitivity and 100% specificity comprises the following:
a) taking a woman's peripheral blood plasma sample;
b) isolating all miRNAs, followed by performing sequence analysis, and the data analysis for the selected 19 miRNAs ordered according to their significance for the in vitro fertilization process result:
iso-hsa-miR-126-3p in SEQ ID NO: 1,
CGTACCGTGAGAAATAATGCGT
iso-hsa-let-7i-5p in SEQ ID NO: 2,
TGTGGTAGTAGTTTGTGCTGTA
iso-hsa-miR-342-3p in SEQ ID NO: 3,
TCTCACACAGAAATCGGACCCGTCT
iso-hsa-let-7b-5p in SEQ ID NO: 4,
TGAGGTAGTAGGTTGTGTGTTA
iso-hsa-miR-132-3p in SEQ ID NO: 5,
TAACAGCCTACAGCCATGGTCGT
iso-hsa-miR-23b-3p in SEQ ID NO: 6,
GCACATTGCCAGGGATTACCACT
iso-hsa-miR-125a-5p in SEQ ID NO: 7,
TCCCTGAGACCTTTTAACCTGT
iso-hsa-miR-584-5p in SEQ ID NO: 8,
TATGGTTTGCCTGGGACTG
iso-hsa-miR-127-3p in SEQ ID NO: 9,
TCGGATCAGTCTGAGCTTGGCTTTT
iso-hsa-miR-101-3p in SEQ ID NO: 10,
TACACTACTGTGATAACTGA
iso-hsa-miR-151a-3p in SEQ ID NO: 11,
TACTAGACTAAAGCTCCTTGAGGT
iso-hsa-miR-92a-3p in SEQ ID NO: 12,
GATTGCACTTGTCCCGGCCTGAA
iso-hsa-let-7b-5p in SEQ ID NO: 13,
TGAGGTAGTATGTTGTGTGG
iso-hsa-miR-589-5p in SEQ ID NO: 14,
TGAGAACCACGTCCGCTCTGAGC
iso-hsa-miR-652-3p in SEQ ID NO: 15,
GATGGCGCCACTAGGGTTGTGA
iso-hsa-miR-1307-3p in SEQ ID NO: 16,
ACTCGGCGTAGCGTCGGTCGTGT
iso-hsa-miR-382-5p in SEQ ID NO: 17,
GAAGTTGTTCGCGGTGGATTC
iso-hsa-let-7b-5p in SEQ ID NO: 18,
TGAGGTAGTAAGTTGTGTGGTAT
iso-hsa-let-7i-5p in SEQ ID NO: 19,
TGAGTTAGTAGTTTGTGCTGTTTT
c) determining the reads number for all 19 miRNA and/or iso-miRNA biomarkers b).

10. The method according to claim 9, wherein the cut off value of 1 for the selected prognostic canonical miRNA and/or iso-miRNA biomarkers from women's peripheral blood plasma on the day of the in vitro fertilization process classifying the miRNA molecules having the value ≥1 to be the molecules predicting successful IVF process, and those having the value <1 to be the molecules predicting IVF process failure, and the cut off value of 5 classifying miRNA molecules having the value ≥5 to be the molecules predicting successful in vitro fertilization process and those having the value <5 to be the molecules predicting the in vitro fertilization process failure.

11. The method according to claim 8, wherein the cut off value of 1 for the selected prognostic canonical miRNA and/or iso-miRNA from the spent blastocyst medium collected once on day 4 to 6 of the embryo's cultivation classifies miRNAs having the value ≥1 to be the molecules predicting a high-competent (high-quality) embryo, and thus the successful in vitro fertilization process, and the values <1 predict a low-competent (low-quality) embryo and thus the in vitro fertilization process failure.


SEQUENCE LISTING (36 sequences)

SEQ ID NO: 1 - RNA, 22 nt, Homo sapiens
  modified_base 3: mod_base = OTHER; note = thymine
  modified_base 8: mod_base = OTHER; note = thymine
  modified_base 15: mod_base = OTHER; note = thymine
  modified_base 18: mod_base = OTHER; note = thymine
  modified_base 22: mod_base = OTHER; note = thymine
  misc_feature 1..22: note = iso-hsa-miR-126-3p
  source 1..22: mol_type = unassigned RNA
cgtaccgtgagaaataatgcgt

SEQ ID NO: 2 - RNA, 22 nt, Homo sapiens
  modified_base 1: mod_base = OTHER; note = thymine
  modified_base 3: mod_base = OTHER; note = thymine
  modified_base 6: mod_base = OTHER; note = thymine
  modified_base 9: mod_base = OTHER; note = thymine
  modified_base 12..14: mod_base = OTHER; note = thymine
  modified_base 16: mod_base = OTHER; note = thymine
  modified_base 19: mod_base = OTHER; note = thymine
  modified_base 21: mod_base = OTHER; note = thymine
  misc_feature 1..22: note = iso-hsa-let-7i-5p
  source 1..22: mol_type = unassigned RNA
tgtggtagtagtttgtgctgta

SEQ ID NO: 3 - RNA, 25 nt, Homo sapiens
  modified_base 1: mod_base = OTHER; note = thymine
  modified_base 3: mod_base = OTHER; note = thymine
  modified_base 14: mod_base = OTHER; note = thymine
  modified_base 23: mod_base = OTHER; note = thymine
  modified_base 25: mod_base = OTHER; note = thymine
  misc_feature 1..25: note = iso-hsa-miR-342-3p
  source 1..25: mol_type = unassigned RNA
tctcacacagaaatcggacccgtct

SEQ ID NO: 4 - RNA, 22 nt, Homo sapiens
  modified_base 1: mod_base = OTHER; note = thymine
  modified_base 6: mod_base = OTHER; note = thymine
  modified_base 9: mod_base = OTHER; note = thymine
  modified_base 13..14: mod_base = OTHER; note = thymine
  modified_base 16: mod_base = OTHER; note = thymine
  modified_base 18: mod_base = OTHER; note = thymine
  modified_base 20..21: mod_base = OTHER; note = thymine
  misc_feature 1..22: note = iso-hsa-let-7b-5p
  source 1..22: mol_type = unassigned RNA
tgaggtagtaggttgtgtgtta

SEQ ID NO: 5 - RNA, 23 nt, Homo sapiens
  modified_base 1: mod_base = OTHER; note = thymine
  modified_base 9: mod_base = OTHER; note = thymine
  modified_base 17: mod_base = OTHER; note = thymine
  modified_base 20: mod_base = OTHER; note = thymine
  modified_base 23: mod_base = OTHER; note = thymine
  misc_feature 1..23: note = iso-hsa-miR-132-3p
  source 1..23: mol_type = unassigned RNA
taacagcctacagccatggtcgt

SEQ ID NO: 6 - RNA, 23 nt, Homo sapiens
  modified_base 6..7: mod_base = OTHER; note = thymine
  modified_base 16..17: mod_base = OTHER; note = thymine
  modified_base 23: mod_base = OTHER; note = thymine
  misc_feature 1..23: note = iso-hsa-miR-23b-3p
  source 1..23: mol_type = unassigned RNA
gcacattgccagggattaccact

SEQ ID NO: 7 - RNA, 22 nt, Homo sapiens
  modified_base 1: mod_base = OTHER; note = thymine
  modified_base 5: mod_base = OTHER; note = thymine
  modified_base 12..15: mod_base = OTHER; note = thymine
  modified_base 20: mod_base = OTHER; note = thymine
  modified_base 22: mod_base = OTHER; note = thymine
  misc_feature 1..22: note = iso-hsa-miR-125a-5p
  source 1..22: mol_type = unassigned RNA
tccctgagaccttttaacctgt

SEQ ID NO: 8 - RNA, 19 nt, Homo sapiens
  modified_base 1: mod_base = OTHER; note = thymine
  modified_base 3: mod_base = OTHER; note = thymine
  modified_base 6..8: mod_base = OTHER; note = thymine
  modified_base 12: mod_base = OTHER; note = thymine
  modified_base 18: mod_base = OTHER; note = thymine
  misc_feature 1..19: note = iso-hsa-miR-584-5p
  source 1..19: mol_type = unassigned RNA
tatggtttgcctgggactg

SEQ ID NO: 9 - RNA, 25 nt, Homo sapiens
  modified_base 1: mod_base = OTHER; note = thymine
  modified_base 6: mod_base = OTHER; note = thymine
  modified_base 10: mod_base = OTHER; note = thymine
  modified_base 12: mod_base = OTHER; note = thymine
  modified_base 17..18: mod_base = OTHER; note = thymine
  modified_base 22..25: mod_base = OTHER; note = thymine
  misc_feature 1..25: note = iso-hsa-miR-127-3p
  source 1..25: mol_type = unassigned RNA
tcggatcagtctgagcttggctttt

SEQ ID NO: 10 - RNA, 20 nt, Homo sapiens
  modified_base 1: mod_base = OTHER; note = thymine
  modified_base 6: mod_base = OTHER; note = thymine
  modified_base 9: mod_base = OTHER; note = thymine
  modified_base 11: mod_base = OTHER; note = thymine
  modified_base 14: mod_base = OTHER; note = thymine
  modified_base 18: mod_base = OTHER; note = thymine
  misc_feature 1..20: note = iso-hsa-miR-101-3p
  source 1..20: mol_type = unassigned RNA
tacactactgtgataactga

SEQ ID NO: 11 - RNA, 24 nt, Homo sapiens
  modified_base 1: mod_base = OTHER; note = thymine
  modified_base 4: mod_base = OTHER; note = thymine
  modified_base 9: mod_base = OTHER; note = thymine
  modified_base 15: mod_base = OTHER; note = thymine
  modified_base 18..19: mod_base = OTHER; note = thymine
  modified_base 24: mod_base = OTHER; note = thymine
  misc_feature 1..24: note = iso-hsa-miR-151a-3p
  source 1..24: mol_type = unassigned RNA
tactagactaaagctccttgaggt

SEQ ID NO: 12 - RNA, 23 nt, Homo sapiens
  modified_base 3..4: mod_base = OTHER; note = thymine
  modified_base 9..10: mod_base = OTHER; note = thymine
  modified_base 12: mod_base = OTHER; note = thymine
  modified_base 20: mod_base = OTHER; note = thymine
  misc_feature 1..23: note = iso-hsa-miR-92a-3p
  source 1..23: mol_type = unassigned RNA
gattgcacttgtcccggcctgaa

SEQ ID NO: 13 - RNA, 20 nt, Homo sapiens
  modified_base 1: mod_base = OTHER; note = thymine
  modified_base 6: mod_base = OTHER; note = thymine
  modified_base 9: mod_base = OTHER; note = thymine
  modified_base 11: mod_base = OTHER; note = thymine
  modified_base 13..14: mod_base = OTHER; note = thymine
  modified_base 16: mod_base = OTHER; note = thymine
  modified_base 18: mod_base = OTHER; note = thymine
  misc_feature 1..20: note = iso-hsa-let-7b-5p
  source 1..20: mol_type = unassigned RNA
tgaggtagtatgttgtgtgg

SEQ ID NO: 14 - RNA, 23 nt, Homo sapiens
  modified_base 1: mod_base = OTHER; note = thymine
  modified_base 12: mod_base = OTHER; note = thymine
  modified_base 17: mod_base = OTHER; note = thymine
  modified_base 19: mod_base = OTHER; note = thymine
  misc_feature 1..23: note = iso-hsa-miR-589-5p
  source 1..23: mol_type = unassigned RNA
tgagaaccacgtccgctctgagc

SEQ ID NO: 15 - RNA, 22 nt, Homo sapiens
  modified_base 3: mod_base = OTHER; note = thymine
  modified_base 12: mod_base = OTHER; note = thymine
  modified_base 17..18: mod_base = OTHER; note = thymine
  modified_base 20: mod_base = OTHER; note = thymine
  misc_feature 1..22: note = iso-hsa-miR-652-3p
  source 1..22: mol_type = unassigned RNA
gatggcgccactagggttgtga

SEQ ID NO: 16 - RNA, 23 nt, Homo sapiens
  modified_base 3: mod_base = OTHER; note = thymine
  modified_base 9: mod_base = OTHER; note = thymine
  modified_base 14: mod_base = OTHER; note = thymine
  modified_base 18: mod_base = OTHER; note = thymine
  modified_base 21: mod_base = OTHER; note = thymine
  modified_base 23: mod_base = OTHER; note = thymine
  misc_feature 1..23: note = iso-hsa-miR-1307-3p
  source 1..23: mol_type = unassigned RNA
actcggcgtagcgtcggtcgtgt

SEQ ID NO: 17 - RNA, 21 nt, Homo sapiens
  modified_base 5..6: mod_base = OTHER; note = thymine
  modified_base 8..9: mod_base = OTHER; note = thymine
  modified_base 15: mod_base = OTHER; note = thymine
  modified_base 19..20: mod_base = OTHER; note = thymine
  misc_feature 1..21: note = iso-hsa-miR-382-5p
  source 1..21: mol_type = unassigned RNA
gaagttgttcgcggtggattc

SEQ ID NO: 18 - RNA, 23 nt, Homo sapiens
  modified_base 1: mod_base = OTHER; note = thymine
  modified_base 6: mod_base = OTHER; note = thymine
  modified_base 9: mod_base = OTHER; note = thymine
  modified_base 13..14: mod_base = OTHER; note = thymine
  modified_base 16: mod_base = OTHER; note = thymine
  modified_base 18: mod_base = OTHER; note = thymine
  modified_base 21: mod_base = OTHER; note = thymine
  modified_base 23: mod_base = OTHER; note = thymine
  misc_feature 1..23: note = iso-hsa-let-7b-5p
  source 1..23: mol_type = unassigned RNA
tgaggtagtaagttgtgtggtat

SEQ ID NO: 19 - RNA, 24 nt, Homo sapiens
  modified_base 1: mod_base = OTHER; note = thymine
  modified_base 5..6: mod_base = OTHER; note = thymine
  modified_base 9: mod_base = OTHER; note = thymine
  modified_base 12..14: mod_base = OTHER; note = thymine
  modified_base 16: mod_base = OTHER; note = thymine
  modified_base 19: mod_base = OTHER; note = thymine
  modified_base 21..24: mod_base = OTHER; note = thymine
  misc_feature 1..24: note = iso-hsa-let-7i-5p
  source 1..24: mol_type = unassigned RNA
tgagttagtagtttgtgctgtttt

SEQ ID NO: 20 - RNA, 21 nt, Homo sapiens
  modified_base 1: mod_base = OTHER; note = thymine
  modified_base 6: mod_base = OTHER; note = thymine
  modified_base 9: mod_base = OTHER; note = thymine
  modified_base 13..14: mod_base = OTHER; note = thymine
  modified_base 16: mod_base = OTHER; note = thymine
  modified_base 18: mod_base = OTHER; note = thymine
  modified_base 21: mod_base = OTHER; note = thymine
  misc_feature 1..21: note = iso-hsa-let-7b-5p
  source 1..21: mol_type = unassigned RNA
tgaggtggtaggttgtgtggt

SEQ ID NO: 21 - RNA, 23 nt, Homo sapiens
  modified_base 1: mod_base = OTHER; note = thymine
  modified_base 21..22: mod_base = OTHER; note = thymine
  misc_feature 1..23: note = iso-hsa-miR-423-5p
  source 1..23: mol_type = unassigned RNA
tgaggggcagagagcgagactta

SEQ ID NO: 22 - RNA, 23 nt, Homo sapiens
  modified_base 1: mod_base = OTHER; note = thymine
  modified_base 5..6: mod_base = OTHER; note = thymine
  modified_base 14: mod_base = OTHER; note = thymine
  modified_base 17: mod_base = OTHER; note = thymine
  modified_base 19..20: mod_base = OTHER; note = thymine
  misc_feature 1..23: note = iso-hsa-miR-21-5p
  source 1..23: mol_type = unassigned RNA
tagcttaccagactgatgttgac

SEQ ID NO: 23 - RNA, 23 nt, Homo sapiens
  modified_base 1: mod_base = OTHER; note = thymine
  modified_base 4: mod_base = OTHER; note = thymine
  modified_base 6: mod_base = OTHER; note = thymine
  modified_base 9: mod_base = OTHER; note = thymine
  modified_base 14: mod_base = OTHER; note = thymine
  misc_feature 1..23: note = iso-hsa-miR-486-5p
  source 1..23: mol_type = unassigned RNA
tcctgtactgagctgcccagaga

SEQ ID NO: 24 - RNA, 22 nt, Homo sapiens
  modified_base 1: mod_base = OTHER; note = thymine
  modified_base 4: mod_base = OTHER; note = thymine
  modified_base 6: mod_base = OTHER; note = thymine
  modified_base 9: mod_base = OTHER; note = thymine
  modified_base 14: mod_base = OTHER; note = thymine
  modified_base 18: mod_base = OTHER; note = thymine
  misc_feature 1..22: note = iso-hsa-miR-486-5p
  source 1..22: mol_type = unassigned RNA
tcctgtactgagctgcctcgag

SEQ ID NO: 25 - RNA, 22 nt, Homo sapiens
  modified_base 3..4: mod_base = OTHER; note = thymine
  modified_base 9..10: mod_base = OTHER; note = thymine
  modified_base 12: mod_base = OTHER; note = thymine
  modified_base 14: mod_base = OTHER; note = thymine
  modified_base 18: mod_base = OTHER; note = thymine
  modified_base 20: mod_base = OTHER; note = thymine
  misc_feature 1..22: note = iso-hsa-miR-25-3p
  source 1..22: mol_type = unassigned RNA
cattgcaattgtctcggtctga

SEQ ID NO: 26 - RNA, 21 nt, Homo sapiens
  modified_base 1: mod_base = OTHER; note = thymine
  modified_base 3..4: mod_base = OTHER; note = thymine
  modified_base 6: mod_base = OTHER; note = thymine
  modified_base 9..18: mod_base = OTHER; note = thymine
  modified_base 20..21: mod_base = OTHER; note = thymine
  misc_feature 1..21: note = iso-hsa-miR-3613-5p
  source 1..21: mol_type = unassigned RNA
tgttgtacttttttttttgtt

SEQ ID NO: 27 - RNA, 23 nt, Homo sapiens
  modified_base 1: mod_base = OTHER; note = thymine
  modified_base 3: mod_base = OTHER; note = thymine
  modified_base 6: mod_base = OTHER; note = thymine
  modified_base 8..10: mod_base = OTHER; note = thymine
  modified_base 13: mod_base = OTHER; note = thymine
  modified_base 17..18: mod_base = OTHER; note = thymine
  modified_base 20: mod_base = OTHER; note = thymine
  misc_feature 1..23: note = iso-hsa-miR-142-3p
  source 1..23: mol_type = unassigned RNA
tgtagtgtttcctaccttatgga

SEQ ID NO: 28 - RNA, 23 nt, Homo sapiens
  modified_base 3: mod_base = OTHER; note = thymine
  modified_base 7: mod_base = OTHER; note = thymine
  modified_base 16: mod_base = OTHER; note = thymine
  modified_base 19..20: mod_base = OTHER; note = thymine
  misc_feature 1..23: note = iso-hsa-miR-664a-5p
  source 1..23: mol_type = unassigned RNA
actggctagggaaaatgattgga

SEQ ID NO: 29 - RNA, 21 nt, Homo sapiens
  modified_base 7: mod_base = OTHER; note = thymine
  modified_base 13: mod_base = OTHER; note = thymine
  modified_base 16..17: mod_base = OTHER; note = thymine
  misc_feature 1..21: note = iso-hsa-miR-151a-3p
  source 1..21: mol_type = unassigned RNA
caagactgaagctccttgagg

SEQ ID NO: 30 - RNA, 22 nt, Homo sapiens
  modified_base 1: mod_base = OTHER; note = thymine
  modified_base 3: mod_base = OTHER; note = thymine
  modified_base 9..10: mod_base = OTHER; note = thymine
  modified_base 12: mod_base = OTHER; note = thymine
  modified_base 20: mod_base = OTHER; note = thymine
  modified_base 22: mod_base = OTHER; note = thymine
  misc_feature 1..22: note = iso-hsa-miR-92a-3p
  source 1..22: mol_type = unassigned RNA
tatcgcacttgtcccggcctgt

SEQ ID NO: 31 - RNA, 20 nt, Homo sapiens
  modified_base 1: mod_base = OTHER; note = thymine
  modified_base 4: mod_base = OTHER; note = thymine
  modified_base 6: mod_base = OTHER; note = thymine
  modified_base 9: mod_base = OTHER; note = thymine
  modified_base 13..14: mod_base = OTHER; note = thymine
  modified_base 16: mod_base = OTHER; note = thymine
  modified_base 18: mod_base = OTHER; note = thymine
  misc_feature 1..20: note = iso-hsa-let-7a-5p
  source 1..20: mol_type = unassigned RNA
tgatgtagtaggttgtatag

SEQ ID NO: 32 - RNA, 24 nt, Homo sapiens
  modified_base 1: mod_base = OTHER; note = thymine
  modified_base 6: mod_base = OTHER; note = thymine
  modified_base 9: mod_base = OTHER; note = thymine
  modified_base 13..14: mod_base = OTHER; note = thymine
  modified_base 16: mod_base = OTHER; note = thymine
  modified_base 18: mod_base = OTHER; note = thymine
  modified_base 21..23: mod_base = OTHER; note = thymine
  misc_feature 1..24: note = iso-hsa-let-7b-5p
  source 1..24: mol_type = unassigned RNA
tgaggtagtaggttgtgtggttta

SEQ ID NO: 33 - RNA, 21 nt, Homo sapiens
  modified_base 7: mod_base = OTHER; note = thymine
  modified_base 11: mod_base = OTHER; note = thymine
  modified_base 18..19: mod_base = OTHER; note = thymine
  modified_base 21: mod_base = OTHER; note = thymine
  misc_feature 1..21: note = iso-hsa-miR-100-5p
  source 1..21: mol_type = unassigned RNA
aacccgtagatccgaacttgt

SEQ ID NO: 34 - RNA, 23 nt, Homo sapiens
  modified_base 4: mod_base = OTHER; note = thymine
  modified_base 9: mod_base = OTHER; note = thymine
  modified_base 22: mod_base = OTHER; note = thymine
  misc_feature 1..23: note = iso-hsa-miR-191-5p
  source 1..23: mol_type = unassigned RNA
caatggaatcccaaaagcagctg

SEQ ID NO: 35 - RNA, 20 nt, Homo sapiens
  modified_base 1: mod_base = OTHER; note = thymine
  modified_base 5..6: mod_base = OTHER; note = thymine
  modified_base 9: mod_base = OTHER; note = thymine
  modified_base 13..14: mod_base = OTHER; note = thymine
  modified_base 16: mod_base = OTHER; note = thymine
  modified_base 18: mod_base = OTHER; note = thymine
  misc_feature 1..20: note = iso-hsa-let-7a-5p
  source 1..20: mol_type = unassigned RNA
tgagttagtaggttgtatag

SEQ ID NO: 36 - RNA, 22 nt, Homo sapiens
  misc_feature 1..22: note = iso-hsa-let-7d 5p
  source 1..22: mol_type = unassigned RNA
  modified_base 6: mod_base = OTHER; note = thymine
  modified_base 9: mod_base = OTHER; note = thymine
  modified_base 13..14: mod_base = OTHER; note = thymine
  modified_base 18: mod_base = OTHER; note = thymine
  modified_base 21..22: mod_base = OTHER; note = thymine
agaggtagtaggttgcgtagtt